(19) United States
(12) Patent Application Publication
(10) Pub. No.: US 2023/0359970 A1
(43) Pub. Date: Nov. 9, 2023

(54) AUTOMATED ESTIMATION AND CAPTURE OF GREENHOUSE GAS EMISSIONS FROM PERFORMANCE OF SOFTWARE-BASED PROCESSES USING CLOUD-BASED INTEGRATION PLATFORM

(71) Applicant: Boomi, LP, Chesterbrook, PA (US)

(72) Inventors: Michael Bachman, Bala Cynwyd, PA (US); John Pflueger, Austin, TX (US)

(21) Appl. No.: 17/735,562

(22) Filed: May 3, 2022

(51) Int. Cl. G06Q 10/06; G16C 20/20

(52) U.S. Cl. CPC G06Q 10/0633; G16C 20/20

(57) ABSTRACT

Automated estimation and capture of greenhouse gas (GHG) emissions from performance of software-based processes using cloud-based integration platform. In an embodiment, an emissions estimation model is stored and associated with a GHG emissions step. The GHG emissions step may be incorporated into a flow path of data through one or more software-based processes. During execution of such a software-based process, data flowing through the GHG emissions step is enhanced by extracting GHG-related data, applying the emissions estimation model to the GHG-related data to estimate GHG emissions, and incorporating the estimated GHG emissions into the data.

### BACKGROUND

### Field of the Invention

The embodiments described herein are generally directed to tracking greenhouse gas (GHG) emissions, and, more particularly, to the automated estimation and capture of GHG emissions, related to the performance of software-based processes, using a cloud-based integration platform.

### Description of the Related Art

In 2018, the Intergovernmental Panel on Climate Change (IPCC) issued a dire warning in a Special Report on the impacts of global warming. According to the report, human activities are estimated to have caused an increase in temperature of roughly 1.0° C. above preindustrial levels. At the present rate, the report states that between 2030 and 2052, Earth's average temperature should increase by 0.5° C., accreting to a total of 1.5° C. of anthropogenic global warming. Unless global warming remains below 1.5° C. (2.0° C. maximum), catastrophic and irreversible ecological outcomes could ensue, with a risk to health, livelihood, food security, water supply, human security, and more.

Global warming is correlated to the production and emission of greenhouse gases, such as carbon dioxide (CO2), methane, nitrous oxide, black carbon, hydrofluorocarbons, and other pollutants. Reducing, eliminating, or reversing the amount of GHG production is critical to maintaining global warming below 1.5° C. Consequently, the accounting of GHG emissions has come under increased regulatory scrutiny. Global enterprises and organizations are taking GHG accounting standards and regulations seriously, in anticipation of policy-based enforcement of zero-emissions requirements.

Currently, manual accounting processes are the norm. While it may be intuitive to humans and the organizations for which they work that GHG emissions must be reduced, it is difficult to grasp all the ways in which GHG emissions can be accounted. Frequently, the investigation of carbon production (i.e., a “carbon footprint”) is either performed at a macro level of gigatons of carbon dioxide (e.g., by the IPCC) or else performed at a micro level by individuals or organizations who must assemble and scour online estimates, documents, spreadsheets, bills, invoices, and the like, to find relevant data and manually tabulate GHG emissions. In addition, the accounting that is adopted by one division of an organization may not be the same as the accounting that is adopted by another division and will likely differ from the accounting that is adopted by other organizations. Needless to say, the manual accounting of GHG emissions across all transactions of an organization is a cumbersome task that cannot currently be performed deterministically, reproducibly, and at scale. For at least these reasons, current approaches are not conducive to automation. Thus, while there are currently guidelines and accounting standards for collecting and reporting GHG emissions, there is no means by which this can be done in an automated, deterministic, reproducible, and scalable manner across an entire organization.

Many transactions of products (e.g., goods or services) can be modeled as a digital business process using software applications. While individual applications may offer means to account for associated GHG emissions, modern transactions typically comprise multiple applications. It is uncommon and unlikely for every application in a modern transaction to enable an accounting of GHG emissions. Furthermore, even if each application did account for GHG emissions, there are no standards, interfaces, and middleware to aggregate GHG data at the level of an entire business process—let alone, an entire organization.

### SUMMARY

Accordingly, systems, methods, and non-transitory computer-readable media are disclosed to for automatically estimating and capturing GHG emissions, related to the performance of software-based processes, using a cloud-based integration platform.

In an embodiment, a method comprises using at least one hardware processor to: store an emissions estimation model that estimates greenhouse gas (GHG) emissions based on GHG-related data; associate the emissions estimation model with a GHG emissions step; and, for each of one or more software-based processes, incorporate the GHG emissions step into a flow path of data through the software-based process, and during execution of the software-based process, for the data flowing through the GHG emissions step incorporated into the software-based process, extract GHG-related data from the data, estimate GHG emissions by applying the emissions estimation model that is associated with the GHG emissions step to the GHG-related data, and enhance the data with the estimated GHG emissions.

Each of the one or more software-based processes may represent a business workflow that acquires the data from at least one data source and sends the enhanced data to at least one destination. Each of the one or more software-based processes may comprise a plurality of steps, including the GHG emissions step, wherein a first one of the plurality of steps comprises acquiring the data from the at least one data source, and wherein a second one of the plurality of steps comprises sending the enhanced data to the at least one destination.

The emissions estimation model may be associated with a scope of emission sources that are modeled by the emissions estimation model, wherein the emissions estimation model estimates only GHG emissions within the associated scope.

The method may further comprise using the at least one hardware processor to: receive a definition of the emissions estimation model; automatically associate an application programming interface with the emissions estimation model, wherein the application programming interface comprises a function that receives GHG-related data as an input, estimates GHG emissions by applying the emissions estimation model to the GHG-related data that was received as input, and returns the estimated GHG emissions; and deploy the emissions estimation model as a microservice. The application programming interface may further comprise a function that returns the definition of the emissions estimation model. The method may further comprise assigning a Uniform Resource Locator (URL) to the emissions estimation model, wherein the URL provides access to the function via a graphical user interface.

Incorporating the GHG emissions step into the flow path of data through the software-based process may comprise, via a graphical user interface: displaying a representation of the software-based process within a virtual canvas in the graphical user interface, wherein the representation of the software-based process comprises visual representations of steps within the software-based process and connections between the visual representations of steps that define the flow path; and receiving a positioning of the GHG emissions step within the displayed representation of the software-based process that defines a position of the GHG emissions step within the flow path. Incorporating the GHG emissions step into the flow path of data through the software-based process may further comprise receiving a configuration of the GHG emissions step through the graphical user interface. The configuration may identify the emissions estimation model from a plurality of available emissions estimation models.

At least one of the one or more software-based processes may be executed on a data stream to enhance the data with estimated GHG emissions in real time.

The at least one hardware processor may be comprised in a cloud infrastructure that dynamically allocates computer system resources to elastically maintain a computing power required to execute the one or more software-based processes. Each of the one or more software-based processes may be comprised in an integration platform in the cloud infrastructure. At least one of the one or more software-based processes may acquire data from a software application or database within the integration platform or external to the integration platform. At least one of the one or more software-based processes may send the enhanced data to a software application within the integration platform or external to the integration platform or stores the enhanced data in a database within the integration platform or external to the integration platform.

The method may further comprise automatically aggregating the estimated GHG emissions over a reporting period.

Enhancing the data may comprise incorporating the estimated GHG emissions into the data or incorporating the estimated GHG emissions into metadata associated with the data.

Any of the methods above may be embodied, individually or in any combination, in executable software modules of a processor-based system, such as a server, and/or in executable instructions stored in a non-transitory computer-readable medium.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates an example infrastructure in which one or more of the processes described herein, may be implemented, according to an embodiment;

FIG. 2 illustrates an example processing system, by which one or more of the processes described herein may be executed, according to an embodiment;

FIG. 3 illustrates a virtual canvas with an example process that incorporates an instance of a GHG emissions step to enhance data, according to an embodiment; and

FIG. 4 illustrates an example architecture of processes that implement automated estimation and capture of GHG emissions, according to an embodiment.

### DETAILED DESCRIPTION

In an embodiment, systems, methods, and non-transitory computer-readable media are disclosed for automatically estimating and capturing GHG emissions, related to the performance of software-based processes, using a cloud-based integration platform. After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

1. Example Infrastructure

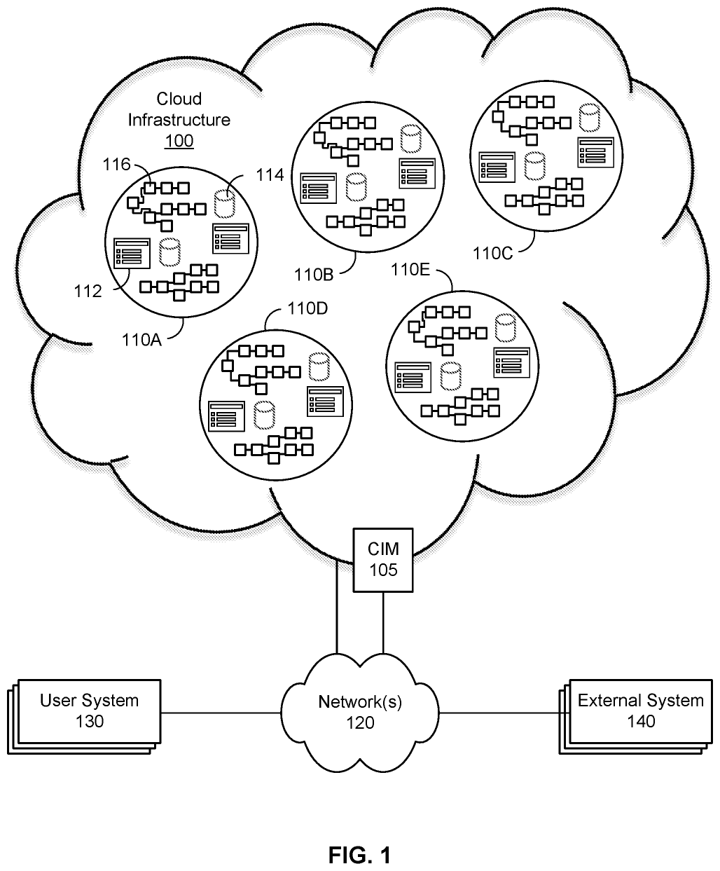

FIG. 1 illustrates an example infrastructure in which one or more of the disclosed processes may be implemented, according to an embodiment. The infrastructure may comprise a cloud infrastructure 100, which provides cloud computing services. Cloud infrastructure 100 may comprise a plurality of computer system resources, including computer processors and data storage. These computer resources may be housed within a single data center or a plurality of data centers distributed over multiple geographic locations.

A cloud infrastructure manager (CIM) 105 may manage cloud infrastructure 100. Cloud infrastructure manager 105 may itself be hosted in cloud infrastructure 100 or may be external to cloud infrastructure 100. Cloud infrastructure manager 105 may dynamically allocate subsets of the available computer system resources in cloud infrastructure 100 to each of a plurality of integration platforms 110 on demand, with upscaling and downscaling of computer system resources according to real-time demand, without direct active management by a user. In other words, cloud infrastructure 100 provides integration platforms as a service (IPaaS). Each integration platform 110 may comprise one or a plurality of applications 112, one or a plurality of databases 114, and/or one or a plurality of processes 116.

Each application 112 may be a cloud-based application that provides one or more services or functions within a business process. Examples of an application 112 include, without limitation, a website, a web application, and a web service, including, for example, applications for Enterprise Resource Planning (ERP), customer relationship management (CRM), scheduling, data storage and backup, invoicing, accounting, payment, business intelligence, supply chain management, human resources management, marketing automation, business process management and automation, and/or the like.

Each database 114 may utilize a pool of data storage within the computer system resources of cloud environment 100 to store structured and/or unstructured data. Structured data may comprise a relational database, such as MySQL™, Oracle™ IBM™, Microsoft SQL™ Access™, PostgreSQL™, MongoDB™, and the like, which store data fields in indexed tables. Unstructured data may include, without limitation, multimedia (e.g., images, video, audio, etc.), text-heavy files, and/or the like, that are stored as files within a file system.

Each process 116 may represent the integration of data between two or more systems. A process 116 may comprise a series of steps that specify logic and transformation requirements for the data to be integrated. Each step may transform, route, and/or otherwise manipulate data to attain an end result from input data. For example, an initial step in a process 116 may retrieve data from one or more data sources, internal steps in a process 116 may manipulate the retrieved data in a specified manner, and a final step in a process 116 may send the manipulated data to one or more specified destinations. The manipulation may comprise any processing of the data, including, without limitation, analyzing, normalizing, altering, updating, and/or enhancing the data. Enhancing the data may comprise adding fields of data or metadata to the data.

Each process 116 may represent a business workflow or a portion of a business workflow or a transaction-level interface between two systems, and comprise, as one or more steps, software modules that process the data within process 116 to implement the business workflow or interface. A business workflow may comprise any myriad of workflows of which an organization may repetitively have need. For example, a business workflow may comprise, without limitation, procurement of parts or materials, manufacturing a product, selling a product, shipping a product, ordering a product, billing, managing inventory or assets, providing customer service, ensuring information security, marketing, onboarding or offboarding an employee, assessing risk, obtaining regulatory approval, reconciling data, auditing data, providing information technology (IT) services, and/or any other workflow that an organization may implement in software.

Each process 116 may communicate, in one or more steps, with one or more applications 112 and/or databases 114 within the same integration platform 110 and/or a different integration platform 110, and/or with one or more applications and/or databases within an external system 140. For example, a step in process 116 may interact with (e.g., retrieve data from or store data to) a database 114 within the same or a different integration platform 110, interact with (e.g., receive data from or send data to) an application 112 within the same or a different integration platform 110, and/or interact with (e.g., receive data from or send data to) an external system 140 via network(s) 120.

Cloud infrastructure 100 may be communicatively connected to one or more networks 120, which may include the Internet. Thus, one or a plurality of user systems 130 and/or one or a plurality of external systems 140 may communicate with cloud infrastructure 100, including with cloud infrastructure manager 105 and/or individual integration platforms 110, via network(s) 120, using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols.

While cloud infrastructure 100 is illustrated as being connected to various systems through a single set of network(s) 120, it should be understood that cloud infrastructure 100 may be connected to the various systems via different sets of one or more networks. For example, cloud infrastructure 100 may be connected to a subset of user systems 130 and/or external systems 140 via the Internet, but may be connected to one or more other user systems 130 and/or external systems 140 via an intranet. Furthermore, while only a single cloud infrastructure 100, several integration platforms 110, and a few user systems 130 and external systems 140 are illustrated, it should be understood that the infrastructure may comprise any number of cloud infrastructures 100, integration platforms 110, user systems 130, and external systems 140.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, Automated Teller Machines (ATMs), and/or the like. Each user system 130 may be used to access an account with cloud infrastructure 100, according to one or more roles or permissions associated with a user of user system 130, who may be identified via authentication. The account may be associated with an organization and used to configure one or more integration platforms 110 for the organization via a graphical user interface provided by cloud infrastructure manager 105. Alternatively, user system 130 may be similarly used to access an account with an external system 140 that interfaces with cloud infrastructure manager 105, to configure one or more integration platforms 110 for the organization via a graphical user interface of external system 140. In this case, cloud infrastructure manager 105 may implement a web service, and external system 140 may interface with the web service via an application programming interface (API). Thus, it should be understood that as used herein and unless stated otherwise, any reference to a “graphical user interface” is a reference to any graphical user interface that is utilized to configure the integration platform(s) 110 associated with an organization, regardless of whether the graphical user interface is generated by cloud infrastructure manager 105, an external system 140, or in some other manner.

In an embodiment, integration platforms 110 are designed and/or tested within cloud infrastructure 100. For example, an organization may authenticate with cloud infrastructure 100 to access one or more tools for designing and/or testing an integration platform within a multi-tenant environment. However, once an integration platform 110 has been constructed, the integration platform 110 may be deployed from cloud infrastructure 100, as a middleware platform, to any production system anywhere in the world. That production system may be cloud-based (e.g., within cloud infrastructure 100 or another cloud) or non-cloud-based (e.g., utilizing dedicated servers) and may be remote or local to the organization. In other words, design-time for an integration platform 110 may occur in the cloud, whereas runtime for the integration platform 110 may be hosted anywhere (e.g., IPaaS), according to the particular organization's implementation.

2. Example Processing Device

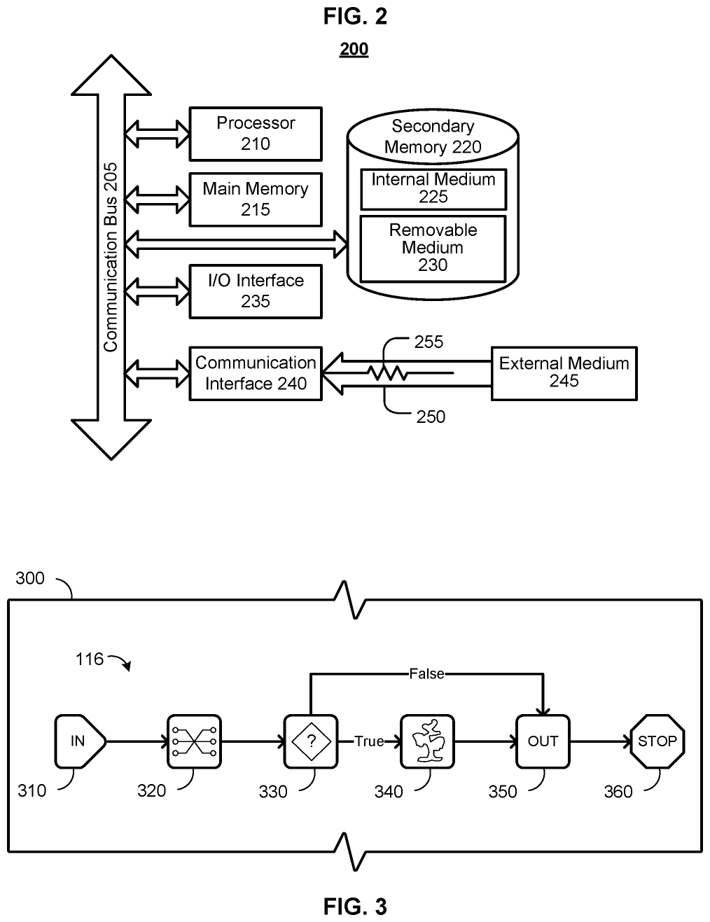

FIG. 2 is a block diagram illustrating an example wired or wireless system 200 that may be used in connection with various embodiments described herein. For example, system 200 may be used as or in conjunction with one or more of the functions, processes, or methods (e.g., to store and/or execute any of the software) described herein, and may represent components of cloud infrastructure 100 (e.g., each of a plurality of servers that form cloud infrastructure 100), user system(s) 130, external system(s) 140, and/or other processing devices described herein. System 200 can be a server or any conventional personal computer, or any other processor-enabled device that is capable of wired or wireless data communication. Other computer systems and/or architectures may be also used, as will be clear to those skilled in the art.

System 200 preferably includes one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 210. Examples of processors which may be used with system 200 include, without limitation, any of the processors (e.g., Pentium™, Core i7™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor 210 may be connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, messaging bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPM), IEEE 696/S-100, event-driven architecture (EDA), publish-and-subscribe (pub-sub), a data mesh, singleton, point-to-point, remote procedure call (RPC) and its derivatives, inter-process communication (IPC), and/or the like, including any other current or future data, messaging, or network technology (e.g., quantum networking).

System 200 preferably includes a main memory 215 and may also include a secondary memory 220. Main memory 215 provides storage of instructions and data for programs executing on processor 210, such as any of the software discussed herein. It should be understood that programs stored in the memory and executed by processor 210 may be written and/or compiled according to any suitable language, including without limitation C/C++, Java, JavaScript, Perl, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 220 is a non-transitory computer-readable medium having computer-executable code (e.g., any of the software disclosed herein) and/or other data stored thereon. The computer software or data stored on secondary memory 220 is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 220 may optionally include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

In alternative embodiments, secondary memory 220 may include other similar means for allowing computer programs or other data or instructions to be loaded into system 200. Such means may include, for example, a communication interface 240, which allows software and data to be transferred from external storage medium 245 to system 200. Examples of external storage medium 245 include an external hard disk drive, an external optical drive, an external magneto-optical drive, and/or the like.

As mentioned above, system 200 may include a communication interface 240. Communication interface 240 allows software and data to be transferred between system 200 and external devices (e.g. printers), networks, or other information sources. For example, computer software or executable code may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 240 are generally in the form of electrical communication signals 255. These signals 255 may be provided to communication interface 240 via a communication channel 250. In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as any of the disclosed software) is stored in main memory 215 and/or secondary memory 220. Computer-executable code can also be received via communication interface 240 and stored in main memory 215 and/or secondary memory 220. Such computer programs, when executed, enable system 200 to perform the various functions of the disclosed embodiments as described elsewhere herein.

In this description, the term “computer-readable medium” is used to refer to any non-transitory computer-readable storage media used to provide computer-executable code and/or other data to or within system 200. Examples of such media include main memory 215, secondary memory 220 (including internal memory 225 and/or removable medium 230), external storage medium 245, and any peripheral device communicatively coupled with communication interface 240 (including a network information server or other network device). These non-transitory computer-readable media are means for providing software and/or other data to system 200.

In an embodiment that is implemented using software, the software may be stored on a computer-readable medium and loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes and functions described elsewhere herein.

In an embodiment, I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

3. Emissions Estimation Model

In an embodiment, a user may define an emissions estimation model in cloud infrastructure 100, for use in an integration platform 110 managed by the user and/or to be used independently of any integration platform 110. For example, a user may utilize the graphical user interface to select an emissions estimation model from a library comprising a plurality of predefined emissions estimation models available to the user's account (e.g., at an account or system level). Alternatively, the user may utilize the graphical user interface to specify (e.g., upload, provide a Uniform Resource Locator (URL) to, build, etc.) a user-defined emissions estimation model. This may be done at an account level or during construction or modification of a process 116. As another alternative, a command-line interface may be provided (e.g., using Secure Shell Protocol (SSH) with cloud infrastructure manager 105) that enables a user to manually define an emissions estimation model (e.g., by specifying variables and/or coefficients).

Regardless of how it is defined, an emissions estimation model may accept one or more GHG-related data fields as input, and output an amount of GHG emissions that is estimated to be produced by activities represented in the GHG-related data. An emissions estimation model may utilize any methodology, including an algebraic expression, a rules-based algorithm, a machine-learning algorithm, a statistics-based algorithm, a physics-based algorithm, and/or the like. Different emissions estimation models may utilize different methodologies. Disclosed embodiments are not constrained by any particular type or methodology of modeling GHG emissions.

Each emissions estimation model may be associated with a “scope,” as defined, for example, by The Greenhouse Gas Protocol of the World Resources Institute. Scope delineates direct and indirect emission sources for GHG accounting and reporting purposes, and is designed to prevent double counting of GHG emissions. There are three scopes defined by The Greenhouse Gas Protocol. Scope 1 encompasses direct GHG emissions occurring from sources that are owned and controlled by the organization, with certain exceptions. Scope 2 encompasses indirect GHG emissions from the generation of purchased electricity (i.e., occurring at the facility where electricity is generated) consumed by the organization. Scope 3 is an optional reporting category that encompasses other indirect GHG emissions, occurring as a consequence of the organization's activities, but not from sources that are owned or controlled by the organization. Examples of GHG emissions in Scope 3 include those occurring as a consequence of extraction and production of purchased materials, transportation of purchased fuels, use of sold products and services, and the like.

Each emissions estimation model may be configured to only calculate a GHG emissions estimate for those activities within the associated scope. Different emissions estimation models may have different scopes, such that each emissions estimation model can be applied individually to GHG-related data to only calculate GHG emissions within its scope, to thereby prevent double counting. To calculate GHG emissions for a plurality of scopes, emissions estimation models, which are each associated with a different one of the plurality of scopes, may all be applied to the input data to calculate GHG emissions for every scope. Alternatively, a single emissions estimation model could be designed to calculate GHG emissions for a plurality of scopes, such that only a single emissions estimation model must be applied to calculate total GHG emissions for the plurality of scopes.

In an embodiment, when an emissions estimation model is created in cloud infrastructure 100, an API is automatically generated for the emissions estimation model and/or associated with the emissions estimation model. For example, this may be performed by cloud infrastructure manager 105. The API may implement standard functions for interacting with the emissions estimation model, thereby enabling the emissions estimation model to be deployed and utilized as a microservice. Access to the emissions estimation model may be restricted by requiring authentication (e.g., using an API key) to utilize one or more functions of the API.

The API functions may comprise application of the emissions estimation model to a set of GHG-related data. For example, a user system 130 or external system 140 may make a remote procedure call to a function of the API of the emissions estimation model, using the GHG-related data as input parameters, and the emissions estimation model may generate an estimate of GHG emissions based on the input parameters and return the estimate to the calling system. Thus, a simple API call can be used to obtain estimates of GHG emissions on demand, thereby reducing the time required to perform an accounting of GHG emissions.

The API functions may also comprise retrieval of the definition of the emissions estimation model itself. For example, a user system 130 or external system 140 may make a remote procedure call to a function of the API of the emissions estimation model, and responsively receive the definition of the emissions estimation model. The definition of the emissions estimation model may identify the methodology, comprise the algorithm or a description of the algorithm, identify the GHG-related data field(s) that are used as input by the emissions estimation model, and/or the like.

Thus, anyone with appropriate permissions may utilize the emissions estimation model and/or acquire the definition of the emissions estimation model. This enables a user to provide access to the emissions estimation model to any third party for utilization, auditing, and/or the like. For example, users could easily share their emissions estimation models with others within their organization, with trading partners, inter-governmental organizations, non-governmental organizations, and/or the like, by simply providing a URL and access to the emissions estimation model.

Alternatively or additionally, an emissions estimation model may be useable via a graphical user interface and/or command-line interface. For example, each emissions estimation model may be associated with a unique URL within cloud infrastructure 100. The URL may provide access to the emissions estimation model via this graphical user interface and/or command-line interface. Access may be restricted by requiring authentication (e.g., username and password, digital certificate, etc.). It should be understood that these interface(s) may be different than the interfaces utilized by users to configure the emissions estimation model. These interface(s) enable a user to interact with the emissions estimation model directly. For example, the user may input GHG-related data into the interface, and the emissions estimation model may generate an estimate of GHG emissions based on the input and display the estimate in the interface. As another example, the user may request the definition of the emissions estimation model, and the interface may display or otherwise provide the definition of the emissions estimation model. It should be understood that information displayed in the graphical user interface may be presented textually or graphically, whereas information displayed in the command-line interface may be presented textually.

Whether as an API, graphical user interface, or command-line interface, the interface for utilizing the emissions estimation model may enable the input of GHG-related data other than the fields used as input by the emissions estimation model. This other GHG-related data may comprise, without limitation, emission reduction goals, formulae, and/or other numbers that may be used for analytics, comparison, and/or the like. For example, in a graphical user interface, emission reduction goals may be graphed against GHG emissions estimated by the emissions estimation model.

Notably, an embodiment in which both an API and a graphical user interface or command-line interface are provided for each emissions estimation model, the emissions estimation model can be utilized and/or otherwise accessed both automatically and manually. This provides flexibility in how the emissions estimation models can be used, and enables each emissions estimation model to be shared and used in myriad applications and contexts.

4. Incorporation of Emissions Estimation Model into Process

In an embodiment, emissions estimation models are integrated into software-based business processes within integration platforms 110. The inventors have discovered that this is an especially effective place for the integration of the emission estimation models, due to the ubiquity of digital representations of transactions as business processes and the ability of an integration platform 110 to join all nodes within a business process. Advantageously, these software-based business processes already model physical processes, including, without limitation, material goods, labor, computations, energy production, movement of objects, and/or the like. Thus, the integration of emissions estimation models into software-based business processes provides an efficient and effective means by which to record, calculate, account, and report GHG emissions at the process level in an automated and scalable manner.

The graphical user interface may comprise an interactive visual model of each integration platform 110 that enables users to intuitively construct each process 116 using visual elements representing the steps of each process 116. An example of such a model is described in U.S. Pat. No. 8,533,661, issued on Sep. 10, 2013, which is hereby incorporated herein by reference as if set forth in full.

Each step in a process 116 represents a task that can be performed on data. For example, an execute step may manipulate or transform the data, a logic step may control the flow of data through process 116, and a connect step may acquire data for process 116 or send data out of process 116. As used herein, it should be understood that “acquiring” data may comprise passively receiving data that have been sent by a data source (e.g., “pushed” by another process 116 from an application 112 or external system 140), actively retrieving data from a data source (e.g., “pulled” from an application 112, database 114, or via an API of an external system 140), or any other manner of acquiring data.

Each process 116 may be graphically represented, on a virtual canvas within the graphical user interface, as a flow path that an instance of data traverses from the point at which it is acquired to the point at which it is sent to one or more destinations. In particular, a representation of a process 116 may be displayed within the virtual canvas as visual representations of each step in process 116 and connections between those visual representations of steps that define the flow path of data through process 116. A user may insert or remove the visual representations of steps from the virtual canvas using, for example, a drag-and-drop input or other form of input. The user may also connect or disconnect the visual representations of steps from each other, as needed to form the desired flow path. It should be understood that the positioning of the visual representation of a step within the representation of process 116 on the virtual canvas defines the position of that step within the flow path. In addition, a user may configure each step, for example, by selecting the visual representation of that step to activate a configuration frame or screen that comprises one or more inputs for specifying parameters of the step.

For ease of understanding, the description of visual representations of a process 116 may simply refer to process 116 itself, and the description of visual representations of steps in a process 116 may simply refer to the steps themselves. It should be understood that, in the context of constructing a process 116 within the graphical user interface, references to the process 116 or its constituent steps are actually references to their visual representations. The actual implementation of a process 116 and its constituent steps are hidden from the user, such that the user does not have to be aware of the actual implementation details. This enables even novice users, without any background in software engineering, to construct processes 116 intuitively. Once a process 116 has been constructed within the virtual canvas and deployed within an integration platform 110, backend functionality (e.g., within cloud infrastructure manager 105 or an external system 140) may automatically convert the visual representation of the process 116 into actual software code (e.g., by configuring and connecting predefined templates of the constituent steps) that can be compiled and executed (e.g., by a virtual machine) within the integration platform 110.

In an embodiment, a plurality of available and commonly used steps are predefined. These predefined steps may be stored (e.g., as templates) in a library that is accessible to all accounts. Thus, a user may simply select a predefined step from the library and insert it at the desired position within the flow path of a process 116. For example, the graphical user interface may comprise a virtual palette that contains graphical representations of the available predefined steps. A user may select a predefined step from the virtual palette and insert it at (e.g., drag it to) a position on the virtual canvas representing its desired position within the flow path, to thereby create an instance of the predefined step within process 116. The user may also configure each particular instance of a predefined step within a process 116, as dictated, for example, by the specific task it is to perform and the specific data on which the task is to be performed.

In an embodiment, the library of predefined steps comprises a GHG emissions step. The GHG emissions step is an execute step that enhances data with an emissions estimate. In particular, the GHG emissions step represents an emissions estimation model that receives data as an input, calculates an estimate of GHG emissions based on at least a relevant subset of the data, referred to herein as “GHG-related data,” and adds the calculated estimate of GHG emissions as a property of the data (e.g., either as a field within the data itself or within metadata associated with the data). In other words, the GHG emissions step applies an emissions estimation model to enhance the data with an estimate of GHG emissions calculated from the GHG-related data.

Each GHG emissions step may be associated with an emissions estimation model, whether by definition or as part of the configuration of the GHG emissions step. While an embodiment could enable a single GHG emissions step to be associated with a plurality of emissions estimation models (e.g., each in a different scope), it will be assumed throughout the present disclosure that a single GHG emissions step is associated with a single emissions estimation model that calculates GHG emissions in a single scope. Advantageously, this enables each emissions estimation model to be utilized atomically in processes 116. For example, the GHG emissions in a plurality of different scopes may be calculated in the same process 116 by simply adding a plurality of GHG emissions steps to the process 116. As another example, a single process 116 or different processes 116 in the same integration platform 116 may utilize different emissions estimation models by incorporating GHG emissions steps that are each associated with a different emissions estimation model.

FIG. 3 illustrates a virtual canvas 300 with an example process 116 that incorporates an instance of an GHG emissions step 340 to enhance data, according to an embodiment. In this particular example, process 116 is dedicated to enhancing data with a GHG emissions estimate along a flow path between a single data source and a single destination. It should be understood that, in practice, GHG emissions step 340 may be incorporated into a more complex process 116 that utilizes other steps to manipulate the data in a plurality of different ways, receive the data from a plurality of data sources, and/or send the data to a plurality of destinations.

In the illustrated example, process 116 comprises an input step 310, a map step 320, a logic step 330, a GHG emissions step 340, an output step 350, and a stop step 360. Process 116 processes instances of data. The data may be related to a business workflow, which may be any type of business workflow. For the purpose of explanation, the example of a manufacturing process of a widget manufacturer will be used throughout. In this case, the data may comprise, for example, a number of widgets produced during a time period of operation.

Input step 310 may acquire input data from a data source. This acquisition may comprise receiving the input data from an application 112, another process 116, or an external system 140, retrieving the input data from an application 112, a database 114, or an external system 140, and/or the like. A user may configure input step 310 to specify the data source and the fields of data to be acquired from the data source. Using the example of the manufacturing process, input step 310 may comprise querying an enterprise resource planning (ERP) system of the manufacturer, implemented as an application 112 or an external system 140, to retrieve a number of widgets that were manufactured by the manufacturer within the past hour.

Map step 320 may map the fields from the input data into fields that are used by subsequent steps. This mapping may receive input data, map the values from native fields in the input data to normalized fields, and output the normalized fields. It should be understood that normalization may be performed for the field names, the data types, and/or the values of the fields. This normalization enables fields to be accessed and utilized consistently by subsequent steps. A user may configure map step 310 to specify the precise mapping. Using the example of the manufacturing process, map step 320 may comprise mapping a field name of the number of widgets (e.g., “number_of_widgets”), as retrieved from the ERP system, to a new normalized field name (e.g., “number_of_products”).

Logic step 330 may determine whether or not the input data comprises GHG-related data. This determination may comprise identifying the presence of specific fields in the input data. Due to the mapping in map step 320, the presence of specific fields can be easily determined by parsing the input data for instances of specific normalized field names that are predefined in advance to represent GHG-related data. Logic step 330 may branch based on the determination. In the illustrated example, if the input data do comprise GHG-related data (i.e., “True”), process 116 flows to GHG emissions step 340. Otherwise, if the input data do not comprise GHG-related data (i.e., “False”), process 116 skips GHG emissions step 340 and flows to output step 350. Using the example of the manufacturing process, logic step 330 may determine whether or not the input data comprises a value for the normalized field name representing GHG-related data (e.g., “number_of_products”).

GHG emissions step 340 receives the input data and applies the emissions estimation model, associated with GHG emissions step 340, to the fields representing the GHG-related data in the input data. The emissions estimation model outputs an estimate of GHG emissions. Using the example of the manufacturing process, GHG emissions step 340 may apply an emissions estimation model for Scope 1 to the GHG-related data (e.g., the value of “number of products”) to generate an estimation of GHG emissions produced by the manufacturing process within the past hour. In this case, the emissions estimation model could comprise an algorithm that is designed to estimate the CO2 emissions output by the widget factory based on the number of widgets that were produced and the rate of production (e.g., number of widgets per hour). The algorithm may be constructed or trained using historical or specified emissions data for each machine involved in the manufacturing process, the widget factory as a whole, and/or by any other appropriate means.

GHG emissions step 340 may incorporate GHG data (e.g., as fields), including the estimated GHG emissions, into the data itself or into metadata that is associated with the data, and output this enhanced data to output step 350. In addition to the estimated GHG emissions, the GHG data may also comprise other data, such as the version number of the emissions estimation model that was applied, update version, type of emissions being estimated (e.g., CO2), an indication of whether or not the estimated GHG emissions are shareable, one or more access restrictions, and/or the like.

Output step 350 may send the data, which may have been enhanced in GHG emission step 340 (i.e., if “True” in logic step 330) or may not have been enhanced (i.e., if “False” in logic step 330), to a destination. This destination may be an application 112, a database 114, an external system 140, and/or the like. A user may configure output step 350 to specify the destination(s) and the fields of data to be sent to the destination(s). Using the example of the manufacturing process, output step 350 may comprise sending the estimated GHG emissions to a customer relationship management (CRM) system of the manufacturer, implemented as an application 112 or an external system 140, so that the carbon footprint of a quantity of widgets purchased by a customer may be calculated (e.g., as a proportion of the estimated GHG emissions corresponding to the purchased quantity of widgets) and reported to that customer.

5. Example Architecture

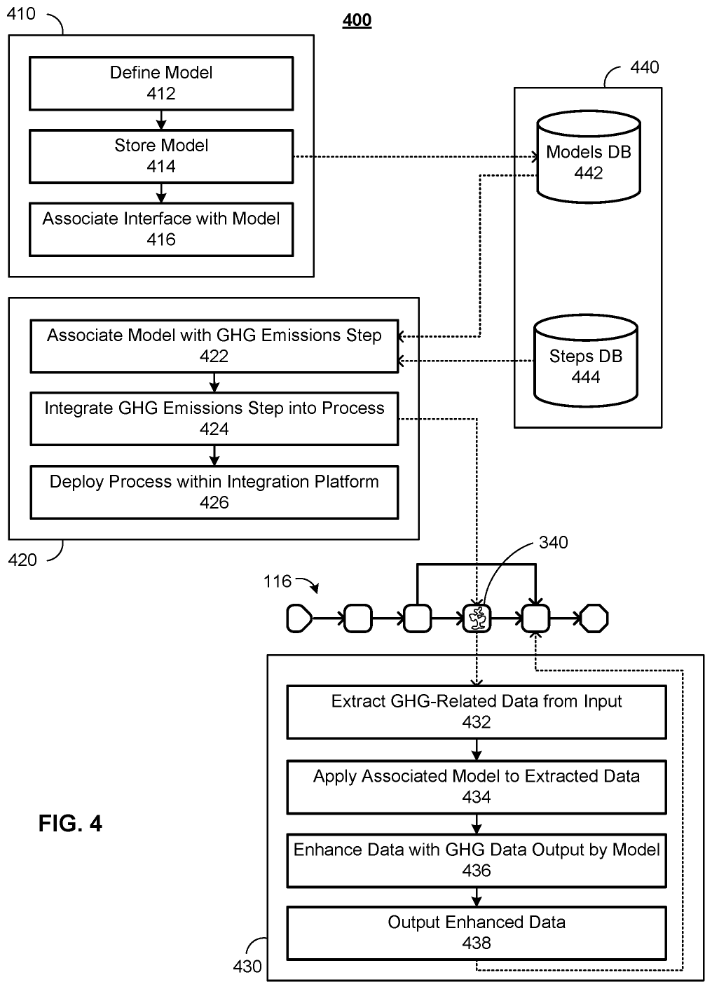

FIG. 4 illustrates an example architecture 400 of processes that implement automated estimation and capture of GHG emissions, according to an embodiment. In particular, architecture 400 may comprise a model-definition process 410, a model-integration process 420, and a model-execution process 430, which may utilize a database 440. It should be understood that processes 410, 420, and 430, which are used to define, deploy, and execute emissions estimation models, are different than processes 116, into which the emissions estimation models may be integrated. Processes 410 and 420 may be implemented by cloud infrastructure manager 105, whereas process 430 may be implemented as a step within a process 116 in an integration platform 110. Database 440 may be managed by cloud infrastructure manager 105 within cloud infrastructure 100 or outside of cloud infrastructure 100.

It should be understood that, although some processes 410-430 may rely on data produced by other processes 410-430, each of processes 410-430 may execute independently of the other processes. In addition, while processes 410-430 are illustrated with a certain arrangement and ordering of subprocesses, each process 410-430 may be implemented with fewer, more, or different subprocesses and a different arrangement and/or ordering of subprocesses. Furthermore, any subprocess, which does not depend on the completion of another subprocess, may be executed before, after, or in parallel with that other independent subprocess, even if the subprocesses are described or illustrated in a particular order.

Model-definition process 410 may be performed for each emissions estimation model that is to be managed within cloud infrastructure 100. In subprocess 412, a new emissions estimation model is defined, as described elsewhere herein. For example, a user may input or upload the parameters defining an emissions estimation model via the graphical user interface or a command-line interface displayed on a user system 130, an external system 140 may send parameters defining an emissions estimation model to cloud infrastructure manager 105 via an API of cloud infrastructure manager 105, cloud infrastructure manager 105 may retrieve parameters defining an emissions estimation model from an external system 140, and/or the like. In subprocess 414, the emissions estimation model, defined in subprocess 412, is stored in a models database 442. In subprocess 416, the emissions estimation model, defined in subprocess 412, is associated with an interface, which may comprise an API, graphical user interface, command-line interface, and/or the like. It should be understood that subprocesses 414 and 416 may be performed in any order or in parallel.

Model-integration process 420 may be performed for each emissions estimation model that is to be used within a process 116. In subprocess 422, an emissions estimation model from models database 442 is associated with a GHG emissions step (e.g., as a template) from steps database 444. This association may be automatic or a manual configuration. For example, each emissions estimation model may be automatically associated with its own GHG emissions step. Alternatively, a user may configure a GHG emissions step to be associated with a particular emissions estimation model from models database 442. In subprocess 424, an instance of the GHG emissions step 340, representing the associated emissions estimation model, is integrated into a process 116. In subprocess 426, the process 116, with the integrated GHG emissions step 340, is deployed within an integration platform 110 to enhance a flow of data with GHG data, as well as potentially perform other tasks on the flow of data.

Model-execution process 430 is performed, during execution of a deployed process 116 in which a GHG emissions step 340 has been integrated, for each iterative execution of GHG emissions step 340. In subprocess 432, GHG-related data is extracted from the data that is provided as input to GHG emissions step 340. In some instances, the input data may consist of (i.e., be coextensive with) the GHG-related data, in which case subprocess 432 may consist of receiving the input data. In subprocess 434, the emissions estimation model that is associated with GHG emissions step 340 (e.g., by virtue of a prior execution of subprocess 422), is applied to the GHG-related data to produce an estimate of GHG emissions. For example, parameter values in the GHG-related data may be used as the values of variables in an algorithm or features in a machine-learning model that predicts GHG emissions. In subprocess 436, the input data is enhanced with GHG data, comprising or consisting of the estimated GHG emissions output by the emissions estimation model. For example, the GHG data may be integrated into the input data and/or into metadata associated with the input data. In subprocess 438, the enhanced data is output by GHG emissions step 340 to the next step, if any, in the flow path of process 116.

6. Example Use Cases

The disclosed embodiments enable fast and convenient deployment of an emissions estimation model within an integration platform 110, for example, as a microservice. In addition, each emissions estimate model may be associated with a GHG emissions step 340 in a library of predefined process steps, such that it can be easily and intuitively incorporated into various software-based business processes within an integration platform 110. When incorporated into a process 116, GHG emissions step 340 automatically calculates GHG data, including an estimate of GHG emissions, using an associated emissions estimation model, and captures this GHG data by enhancing the data flowing through GHG emissions step 340 with the GHG data. Advantageously, the integration of emissions estimation models into processes 116 in this manner enables GHG emissions to be calculated and captured automatically, deterministically, reproducibly, and at scale, in real time, as data flows through integration platform 110.

For example, a process 116 could be executed on a constant or continuous data stream to enhance the data with GHG data in real time. In this case, cloud infrastructure 100 may dynamically allocate computer system resources to elastically maintain the computing power required to process the data in real time, regardless of the amount or rate of data. Thus, the data may be automatically enhanced with GHG data at any scale supportable by cloud infrastructure 100.

In addition, disclosed embodiments pave the way for the creation of standards and the adoption of such standards across any industry. For the purposes of explanation, a few example use cases will be described. However, it should be understood that this is just a small sample and that there are a vast number of other use cases and contexts that will benefit from disclosed embodiments.

In a first exemplary use case, disclosed embodiments may perform transaction-based carbon accounting for downstream sales of products (e.g., goods and/or services). In this case, a workflow, comprising GHG emissions step 340, may be added to an existing process 116 that implements order entry and fulfillment, to calculate and capture an estimated carbon footprint for the products that are provided to customers. The estimated carbon footprint could be provided to the customers so that they may account for their own Scope-3 carbon footprints.

In a second exemplary use case, disclosed embodiments may perform transaction-based carbon accounting for upstream sales of products. In this case, a workflow, comprising GHG emissions step 340, may be added to an existing process 116 that implements procurement, to calculate and capture an estimated carbon footprint for the products that are purchased from suppliers. The procurer may utilize the estimated carbon footprint to account for its Scope-3 carbon footprint.

In a third exemplary use case, disclosed embodiments may perform real-time carbon accounting for specific information technology (IT) operations or services. In this case, a workflow, comprising GHG emissions step 340, may be added to an existing process 116 that implements specific operation(s) performed as part of an IT service, to calculate and capture an estimated carbon footprint for the specific operation(s). The resulting carbon footprint can be used for internal carbon accounting and assignment (e.g., implementing an internal tax on carbon for activities) and/or communicated to external entities that receive the benefits of the specific operation(s).

In a fourth exemplary use case, disclosed embodiments may perform regularly occurring carbon accounting for specific activities within an enterprise. Many commercial and public organizations are responsible for reporting climate-related performance on a regular cadence. Today, this cadence is generally annual. However, expectations are that the cadence will increase—most likely to quarterly at first, and then potentially to monthly or weekly. Current accounting practices rely heavily on manual activities, resulting in significant latencies between the end of a reporting period and the completion of the report. The disclosed embodiments may be utilized to automatically calculate and capture GHG data, so as to satisfy the increasing reporting frequencies and reduce reporting latencies. In particular, the incorporation of GHG emissions step 340 into the software-based business processes 116 of an organization enables GHG emissions to be automatically captured. These captured GHG emissions may be automatically aggregated over each reporting period, for example, by a process 116 that is configured to aggregate GHG emissions from one or more data sources (e.g., a database 114 within the same integration platform 110). The aggregate GHG emissions may then be automatically incorporated into a report within a graphical user interface (e.g., accessed by a user system 130), electronic document (e.g., in the format of eXtensible Markup Language (XML), JavaScript Object Notation (JSON), Portable Document Format (PDF), a spreadsheet), and/or the like. The aggregate GHG emissions may be provided at one or a plurality of levels of granularity (e.g., for the entire reporting period, per business process, etc.).

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A method comprising using at least one hardware processor to:
store an emissions estimation model that estimates greenhouse gas (GHG) emissions based on GHG-related data;
associate the emissions estimation model with a GHG emissions step; and,
for each of one or more software-based processes,
incorporate the GHG emissions step into a flow path of data through the software-based process, and
during execution of the software-based process, for the data flowing through the GHG emissions step incorporated into the software-based process,
extract GHG-related data from the data,
estimate GHG emissions by applying the emissions estimation model that is associated with the GHG emissions step to the GHG-related data, and
enhance the data with the estimated GHG emissions.

2. The method of claim 1, wherein each of the one or more software-based processes represents a business workflow that acquires the data from at least one data source and sends the enhanced data to at least one destination.

3. The method of claim 2, wherein each of the one or more software-based processes comprises a plurality of steps, including the GHG emissions step, wherein a first one of the plurality of steps comprises acquiring the data from the at least one data source, and wherein a second one of the plurality of steps comprises sending the enhanced data to the at least one destination.

4. The method of claim 1, wherein the emissions estimation model is associated with a scope of emission sources that are modeled by the emissions estimation model, and wherein the emissions estimation model estimates only GHG emissions within the associated scope.

5. The method of claim 1, further comprising using the at least one hardware processor to:
receive a definition of the emissions estimation model;
automatically associate an application programming interface with the emissions estimation model, wherein the application programming interface comprises a function that receives GHG-related data as an input, estimates GHG emissions by applying the emissions estimation model to the GHG-related data that was received as input, and returns the estimated GHG emissions; and
deploy the emissions estimation model as a microservice.

6. The method of claim 5, wherein the application programming interface further comprises a function that returns the definition of the emissions estimation model.

7. The method of claim 5, further comprising assigning a Uniform Resource Locator (URL) to the emissions estimation model, wherein the URL provides access to the function via a graphical user interface.

8. The method of claim 1, wherein incorporating the GHG emissions step into the flow path of data through the software-based process comprises, via a graphical user interface:
displaying a representation of the software-based process within a virtual canvas in the graphical user interface, wherein the representation of the software-based process comprises visual representations of steps within the software-based process and connections between the visual representations of steps that define the flow path; and
receiving a positioning of the GHG emissions step within the displayed representation of the software-based process that defines a position of the GHG emissions step within the flow path.

9. The method of claim 8, wherein incorporating the GHG emissions step into the flow path of data through the software-based process further comprises receiving a configuration of the GHG emissions step through the graphical user interface.

10. The method of claim 9, wherein the configuration identifies the emissions estimation model from a plurality of available emissions estimation models.

11. The method of claim 1, wherein at least one of the one or more software-based processes is executed on a data stream to enhance the data with estimated GHG emissions in real time.

12. The method of claim 1, wherein the at least one hardware processor is comprised in a cloud infrastructure that dynamically allocates computer system resources to elastically maintain a computing power required to execute the one or more software-based processes.

13. The method of claim 12, wherein each of the one or more software-based processes is comprised in an integration platform in the cloud infrastructure.

14. The method of claim 13, wherein at least one of the one or more software-based processes acquires data from a software application or database within the integration platform or external to the integration platform.

15. The method of claim 13, wherein at least one of the one or more software-based processes sends the enhanced data to a software application within the integration platform or external to the integration platform or stores the enhanced data in a database within the integration platform or external to the integration platform.

16. The method of claim 1, further comprising automatically aggregating the estimated GHG emissions over a reporting period.

17. The method of claim 1, wherein enhancing the data comprises incorporating the estimated GHG emissions into the data.

18. The method of claim 1, wherein enhancing the data comprises incorporating the estimated GHG emissions into metadata associated with the data.

19. A system comprising:
at least one hardware processor; and
one or more software modules that are configured to, when executed by the at least one hardware processor,
store an emissions estimation model that estimates emissions based on emissions-related data,
associate the emissions estimation model with an emissions step, and,
for each of one or more software-based processes,
incorporate the emissions step into a flow path of data through the software-based process, and
during execution of the software-based process, for the data flowing through the emissions step incorporated into the software-based process,
extract emissions-related data from the data,
estimate emissions by applying the emissions estimation model that is associated with the emissions step to the emissions-related data, and
enhance the data with the estimated emissions.

20. A non-transitory computer-readable medium having instructions stored therein, wherein the instructions, when executed by a processor, cause the processor to:
store an emissions estimation model that estimates emissions based on emissions-related data;
associate the emissions estimation model with an emissions step; and,
for each of one or more software-based processes,
incorporate the emissions step into a flow path of data through the software-based process, and
during execution of the software-based process, for the data flowing through the emissions step incorporated into the software-based process,
extract emissions-related data from the data,
estimate emissions by applying the emissions estimation model that is associated with the emissions step to the emissions-related data, and
enhance the data with the estimated emissions.
